(19) United States
(12) Patent Application Publication
(10) Pub. No.: US 2022/0154124 A1
(43) Pub. Date: May 19, 2022

(54) BIOREACTOR WITH VOLUME-ADJUSTABLE PACKING LAYER

(71) Applicant: GWANGJU INSTITUTE OF SCIENCE AND TECHNOLOGY, Gwangju (KR)

(72) Inventors: In Seop CHANG, Gwangju (KR); Nu Lee JANG, Gwangju (KR)

(21) Appl. No.: 17/432,360

(22) PCT Filed: Apr. 22, 2020
(86) PCT No.: PCT/KR2020/005272

§ 371 Date: Aug. 19, 2021

(30) Foreign Application Priority Data
May 10, 2019 (KR) 10-2019-0055111

(51) Int. Cl. C12M 1/12; C12M 1/00; C12M 1/34

(52) U.S. Cl. CPC C12M 25/18; C12M 23/02; C12M 41/34; C12M 29/24; C12M 41/26; C12M 29/06

(57) ABSTRACT

There is provided a bioreactor including: a chamber unit having an accommodating space therein; an upper fixing member and a lower fixing member that are disposed at respective planes perpendicular to a central axis of the chamber unit inside the chamber unit and are movable in a central-axis direction of the chamber unit; and a filling layer that is formed in a space between the upper fixing member and the lower fixing member inside the chamber unit and contains a plurality of carriers.

### TECHNICAL FIELD

The present invention relates to a bioreactor that enables a microbiological culture.

### BACKGROUND ART

A microorganism has various enzymes in its body and, thus, can convert substrate substances into high value products or can decompose pollutants. A bioreactor has been used to culture massive microorganisms and enzymes or to increase a biological reaction rate. In particular, improvement in physical performance in a bioreactor or an increase in production yield and production speed of a target metabolite by adding a chemical or biological characteristic have been actively studied.

A bioreactor is a device that converts a reactant into a product by using microorganisms or enzymes as catalysts, and the bioreactor is also referred to as a biological reaction device. An organism decomposes a substrate substance and compounds a final metabolite in its body by using various types of enzymes. Here, a decomposition or composition reaction thereof is usually performed at room temperature and pressure, and a reactant and a product vary depending on an enzyme and a metabolic pathway held in a microorganism.

A biological reaction has advantages in that there is no need to use an expensive metal catalyst in a reaction with the same reactant and product or a high-temperature and high-pressure physicochemical process, and waste produced after proceeding of processes also has low environmental toxicity.

Hence, the bioreactor has advantages in that not only can resource and energy be saved but also smoke, harmful substance, or noise pollution is not produced.

Examples of a biocatalyst used in a bioreactor include an enzyme, a microorganism, an animal cell, and a plant cell, and the biocatalyst is suspended or immobilized to be used. Increases in conversion rate of a substrate into a product, product yield, product density, and productivity are important to be considered in a design of a bioreactor.

A bioreactor using conventional carriers has problems in that the carriers generally move randomly and float in a liquid in the reactor; in this case, biofilms formed on the carriers are desorbed due to collision between carriers; as a result, density of biocatalysts in the reactor is also decreased.

In particular, in a case of a gas fermentation process using a gas, a substrate gas is supplied in a liquid phase from a bottom of a reactor. In this case, liquid turbulence and shearing force increase due to a high gas flow speed; as a result, shaking of carriers increases, and thus biofilms are more desorbed. In addition, carriers float to an upper end of the reactor and are separated from the liquid. Hence, substrate gas converting performance of the process is decreased, and a problem is highly likely to arise physically in a process operation.

### SUMMARY OF INVENTION

### Technical Problem

An object of the present invention is to provide a bioreactor including a filling layer of which a volume is adjustable.

Another object of the present invention is to provide a method for culturing microorganisms using the bioreactor.

### Solution to Problem

According to an aspect of the present invention, there is provided a bioreactor including: a chamber unit having an accommodating space therein; an upper fixing member and a lower fixing member that are disposed at respective planes perpendicular to a central axis of the chamber unit inside the chamber unit and are movable in a central-axis direction of the chamber unit; and a filling layer that is formed in a space between the upper fixing member and the lower fixing member inside the chamber unit and contains a plurality of carriers.

In an embodiment of the present invention, the filling layer may have a volume which is adjustable by adjusting a position of the upper fixing member and/or the lower fixing member. In the embodiment of the present invention, the bioreactor may further include a fixing-member adjusting unit.

In the embodiment of the present invention, the bioreactor may further include a gas supply unit.

In the embodiment of the present invention, the bioreactor may further include an inlet and an outlet.

In the embodiment of the present invention, the bioreactor may further include a gas supply channel and a gas outflow channel.

In the embodiment of the present invention, the bioreactor may further include a gas re-circulation line that is branched from the gas outflow channel and is connected to the gas supply channel.

In the embodiment of the present invention, the bioreactor may further include a sensor.

In the embodiment of the present invention, the sensor may measure any one selected from the group consisting of pH, oxidation-reduction potential, dissolved oxygen, turbidity, a shape, and a combination thereof.

In the embodiment of the present invention, the bioreactor may further include a liquid distributing unit that is positioned at an upper surface inside the chamber unit.

According to another aspect of the present invention, there is provided a bioreactor including: a chamber unit having an accommodating space therein; a partition member that is positioned to be coaxial with a central axis of the chamber unit inside the chamber unit and is separated from a side wall of the chamber unit to form a circulation part; an upper fixing member and a lower fixing member that are disposed at respective planes perpendicular to the central axis of the chamber unit and are positioned inside the partition member; and a filling layer that is formed in an inner space of the partition member and a space between the upper fixing member and the lower fixing member and contains a plurality of carriers. The circulation part is formed in a separated space between the side wall of the chamber unit and the partition member.

According to still another aspect of the present invention, there is provided a bioreactor including: a chamber unit having an accommodating space therein; a partition member that is positioned to be coaxial with a central axis of the chamber unit inside the chamber unit and is separated from a side wall of the chamber unit to form a circulation part; an upper fixing member and a lower fixing member that are disposed at respective planes perpendicular to the central axis of the chamber unit and are positioned in a space between an outer wall of the partition member and an inner wall of the chamber unit; a filling layer that is formed in a space between the inner wall of the chamber unit and the outer wall of the partition member and a space between the upper fixing member and the lower fixing member and contains a plurality of carriers. The circulation part is formed in a space inside the partition member.

According to still another aspect of the present invention, there is provided a method for culturing microorganisms by using the bioreactor provided in the embodiments of the present invention.

In another embodiment of the present invention, the method for culturing microorganisms may be performed by any one method of batch culture, continuous culture, or semibatch culture.

### Advantageous Effects of Invention

According to an embodiment of the present invention, there is provided a bioreactor with a filling layer having an adjustable volume.

The bioreactor provided according to the embodiment of the present invention confines carriers inside the filling layer during an operation thereof, and thereby frequency of desorption of biofilms due to collision between carriers and liquid turbulence during the operation of the bioreactor are decreased such that density of biocatalysts inside the reactor is inhibited from decreasing.

In particular, the bioreactor according to the embodiment of the present invention can inhibit a decrease in frequency of reaction due to desorption of biofilms caused by a gas supplied at a high flow speed or due to upward floating of carriers.

In addition, as the filling layer has the adjustable volume, overall metabolism performed inside the bioreactor is adjustable by using an appropriate amount of carriers only with a simple process of adjusting the volume of the bioreactor, and thus there is an advantage of an increase in yield of a target product from a reactant.

The effects of the present invention are construed not to be limited to the above-described effects but to include every effect that can be derived from the configurations of the present invention described in Description of Embodiments or Claims of the present invention.

### BRIEF DESCRIPTION OF DRAWINGS

a and b of FIG. 1 are schematic views of bioreactors according to embodiments of the present invention.

FIG. 2 is an enlarged view of a top surface of an upper fixing member and a lower fixing member according to the embodiments of the present invention.

FIGS. 3 and 4 and a and b of FIG. 5 are schematic views of bioreactors according to another embodiment of the present invention.

FIG. 6 is a graph illustrating cell growth and a production pattern of a product during a microbiological culture according to an embodiment of the present invention.

### DESCRIPTION OF EMBODIMENTS

Hereinafter, the present invention will be described with reference to the accompanying drawings. However, the present invention can be realized as various different examples, thus not being limited to the embodiments described here. Besides, a part unrelated to the description is omitted from the drawings in order to clearly describe the present invention, and similar reference signs are assigned to similar parts through the entire specification.

In the entire specification, a case where a certain part “is connected to (accesses, is in contact with, or is coupled to)” another part includes not only a case where the parts are “directly connected” to each other, but also a case where the parts are “indirectly connected” to each other with another member interposed therebetween. In addition, a case where a certain part “comprises” a certain configurational element means that another configurational element is not excluded but can be further included, unless specifically described otherwise.

Terms used in this specification are only used to describe a specific embodiment and are not intentionally used to limit the present invention thereto.

FIG. 1 illustrates schematic views of bioreactors 1a and 1b according to embodiments of the present invention.

With reference to a) of FIG. 1, a bioreactor 1a includes: a chamber unit 100 having an accommodating space therein; an upper fixing member 210 and a lower fixing member 220 that are disposed at respective planes perpendicular to a central axis of the chamber unit 100 inside the chamber unit 100 and are movable in a central-axis direction of the chamber unit 100; and a filling layer 200 that is positioned in a space between the upper fixing member 210 and the lower fixing member 220 inside the chamber unit 100 and contains a plurality of carriers 700.

The chamber unit 100 forms the external appearance of the bioreactor 1a and has a function of accommodating an interior configuration of the bioreactor 1a.

The chamber unit 100 can be made of a sterilizable material such as one selected from the group consisting of glass, engineering plastics, ceramic, polymer, stainless steel, other alloys, and a combination thereof, and the chamber unit can be made of stainless steel, for example; however, the material thereof is not limited thereto.

The bioreactor 1a of the present invention includes the upper fixing member 210 and the lower fixing member 220.

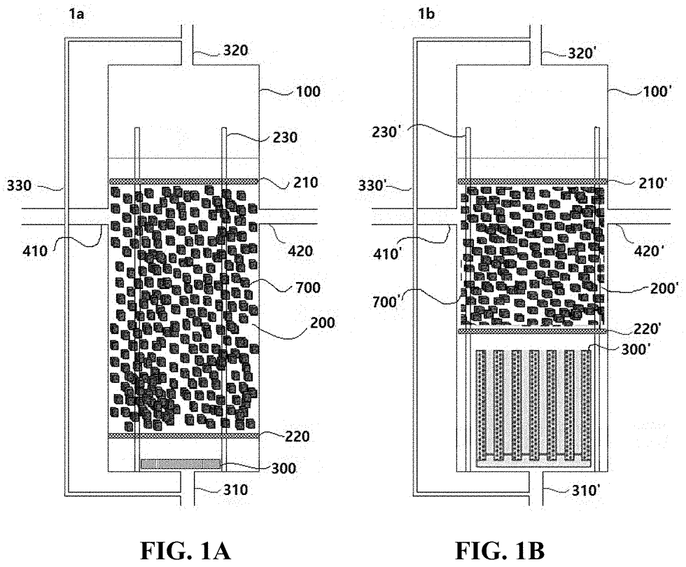

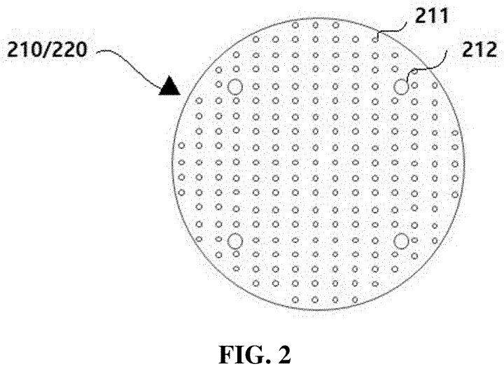

FIG. 2 is an enlarged view of a top surface of the upper fixing member 210 and/or the lower fixing member 220 according to the embodiments of the present invention.

With reference to FIG. 2, the upper fixing member 210 and the lower fixing member 220 can have a plurality of holes such as a first hole 211 and a second hole 212, which have different sizes from each other.

The plurality of holes are used for different purposes depending on a size thereof. The first hole 211 can be relatively smaller than a size of the second hole 212. The first hole 211 can be smaller than a size of a carrier 700 contained in the filling layer 200, and the first hole 211 can have two or more sizes.

The first hole 211 has a diameter in a range smaller than the size of the carrier 700 contained in the filling layer 200, for example may be of 1 mm to 10 mm, but it is not limited thereto.

For example, since the first hole 211 of the upper fixing member 210 and the lower fixing member 220 can be smaller than the size of the carrier 700 contained in the filling layer 200, the carrier 700 does not pass and does not move through the upper fixing member 210 and the lower fixing member 220; however, the carrier can pass through a culture medium and gas inside the chamber unit 100. Consequently, the first hole 211 enables the gas and the culture medium to circulate inside the chamber unit 100.

The second hole 212 can be relatively larger than the size of the first hole 211. The second hole 212 can be omitted from a structure of the upper fixing member 210 and the lower fixing member 220.

The upper fixing member 210 and the lower fixing member 220 has a function of fixing the carriers 700 in the filling layer 200 formed inside the chamber unit 100 and limiting movement of the carrier 700.

In order to effectively limit the movement of the carrier 700, it is preferable that a gap between a side portion of the upper fixing member 210 and the lower fixing member 220 and an inner wall of the chamber unit 100 be smaller than a diameter of the carrier 700. For example, a cross section of the filling layer 200 has the same width and shape as a cross section of the inside of the upper fixing member 210 and the lower fixing member 220, and there may be little gap between the side portion of the upper fixing member 210 and the lower fixing member 220 and an inner wall of the chamber unit 100.

The upper fixing member 210 and the lower fixing member 220 can be made of a sterilizable material such as one selected from the group consisting of glass, engineering plastics, ceramic, polymer, stainless steel, other alloys, and a combination thereof, and the upper fixing member and the lower fixing member can be made of stainless steel, for example; however, the material thereof is not limited thereto. In addition, the upper fixing member 210 and the lower fixing member 220 can be made of the same material.

The bioreactor 1a can further include a fixing-member adjusting unit 230. The upper fixing member 210 and the lower fixing member 220 can move in both directions of a central-axis direction of the chamber unit 100 by the fixing-member adjusting unit 230.

The fixing-member adjusting unit 230 has a function of moving and supporting the upper fixing member 210 and the lower fixing member 220 inside the chamber unit 100. The fixing-member adjusting unit 230 can be a unit that can be connected to an upper portion of the upper fixing member 210 and a lower portion of the lower fixing member 220 inside the chamber unit 100 and that can move through the upper fixing member 210 and the lower fixing member 220, for example.

The fixing-member adjusting unit 230 can have any one shape selected from the group consisting of a tube shape, a bar shape, a wire shape, a rectangular parallelepiped shape, a plate shape, and a combination thereof; however, the shape of the fixing-member adjusting unit is not limited thereto. For example, the fixing-member adjusting unit 230 can have the bar shape so as to penetrate through the upper fixing member 210 and the lower fixing member 220.

In the embodiment of the present invention, upper surfaces of the upper fixing member 210 and/or the lower fixing member 220 can have the second hole 212 having the same shape and width as a cross section of the fixing-member adjusting unit 230, and the fixing-member adjusting unit 230 can penetrate through second holes of the upper fixing member 210 and the lower fixing member 220.

The filling layer 200 contains the carriers 700, and a volume and a position of the filling layer are determined depending on movement of the movable upper fixing member 210 and/or the movable lower fixing member 220.

The carrier 700 can have any one shape of a cube shape, a tetrahedron shape, a spherical shape, a tube shape, and a combination thereof and can have the cube shape, for example. The carrier 700 can have a volume in a range of 0.5 cm³ to 2 cm³, for example, 1 cm³, which is larger than the diameter of the first hole 211; however, the volume of the carrier is not limited thereto.

Examples of a material of the carrier 700 can be any one selected from the group consisting of polyurethane, polyethylene, polyvinylchloride, polyvinyl alcohol, polypropylene, polysulfonate, polycarbonate, polyamide, polyacetal, polyethylene terephthalate, modified polyphenylene oxide, cellulose, porous glass, ceramic, clay ball, zeolite, and a combination thereof, and the carrier can be made of polyurethane, for example; however, the material thereof is not limited thereto.

In the embodiment of the present invention, the density of the filling layer 200 containing the carriers 700 can be a controlling factor used to control a yield of product. When the density of the filling layer 200 containing the carriers 700 is too low, if a gas supply unit 300 supplies gas at a high flow speed, rising of gas can result in an increase in shaking of the culture medium and the carriers and an increase in desorption of biofilms, and thus the carriers 700 can float to an upper end of the reactor. As a result, an operation of a process can be difficult to perform. Conversely, when the density of the filling layer 200 containing the carriers 700 is too high, circulation of the gas and the culture medium can be hindered. Hence, the filling layer 200 containing the carriers 700 needs to have appropriate density.

The bioreactor 1a of the present invention is advantageous in that the volume of the filling layer 200 can be adjusted only with simple adjustment performed by causing the fixing-member adjusting unit 230 to move the upper fixing member 210 and the lower fixing member 220 such that the density of the filling layer 200 containing the carriers 700 can be adjusted.

The bioreactor 1a can further include an inlet 410 and an outlet 420.

In the embodiment of the present invention, the inlet 410 and the outlet 420 can be positioned at a side surface of the chamber unit 100.

In the embodiment of the present invention, the inlet 410 can be used as a passage through which a liquid, such as a culture medium, which is needed for an operation of the bioreactor 1a can be injected into the chamber unit 100.

For example, when the bioreactor 1a is operated by the batch culture, the inlet 410 can be used as a passage through which a culture medium is injected at an initial operation of the bioreactor 1a and can be closed during the operation thereof.

For example, when the bioreactor 1a is operated by the continuous culture, the inlet 410 can be used as a passage through which a culture medium is continuously injected during the operation of the bioreactor 1a.

In the embodiment of the present invention, the outlet 420 can be used as a passage through which a product, such as a fermented solution, which has been produced after the operation of the bioreactor 1a can be discharged outside the chamber unit 100.

For example, when the bioreactor 1a is operated by the continuous culture, the outlet 420 can be used as a passage through which a fermented solution can be continuously discharged outside during the operation of the bioreactor 1a. Hence, since a culture medium is continuously injected through the inlet 410 and the fermented solution is continuously discharged through the outlet 420, a constant amount of culture medium is maintained inside the chamber unit 100, and a new culture medium is supplied such that microorganisms are not killed and can be continuously cultured.

In the embodiment of the present invention, the bioreactor 1a can further include a gas supply channel 310 and a gas outflow channel 320.

In the embodiment of the present invention, the gas supply channel 310 can be positioned at a lower side of the chamber unit 100, and the gas outflow channel 320 can be positioned at an upper side of the chamber unit 100.

In the embodiment of the present invention, the gas supply channel 310 supplies gas and has a function of moving a part of gas inside the chamber unit 100 from the lower side to the upper side of the chamber unit 100.

In the embodiment of the present invention, gas supplied through the gas supply channel 310 can be any one selected from the group consisting of CO, CO₂, H₂, O₂, and a combination thereof, and the gas can be syngas obtained by mixing CO, CO₂, and H₂, for example.

For example, new gas is supplied through the gas supply channel 310, the supplied gas can pass through the gas supply unit 300 in the chamber unit 100 and can pass through the lower fixing member 220 to come into contact with the carriers 700 in the filling layer 200. Subsequently, the gas can pass through the upper fixing member 210, then through the gas outflow channel 320 and can be discharged outside the chamber unit 100.

Since new gas is continuously supplied through the gas supply channel 310 and the gas is removed through the gas outflow channel 320, a constant amount of gas is maintained inside the chamber unit 100 such that a fermentation reaction can proceed.

The bioreactor 1a can further include the gas supply unit 300.

The gas supply unit 300 has functions of converting the gas supplied through the gas supply channel 310 into gas having a fine bubble shape and supplying the fine bubble-shaped gas into the chamber unit 100.

The gas supply unit 300 can include a material with micropores, such as one selected from the group consisting of a ceramic filter, a ceramic membrane, a hollow fiber membrane, a flat membrane, a porous stainless steel plate, and a combination thereof, and the gas supply unit can include the ceramic filter, for example.

The gas supply unit 300 can have any one shape selected from the group consisting of a tube shape, a bar shape, a wire shape, a hexahedral shape, a plate shape, and a combination thereof, and the shape of the gas supply unit can be the bar shape, for example.

In the embodiment of the present invention, one or more gas supply units 300 can be provided and can be positioned in a plane direction perpendicular to the central axis of the chamber unit 100.

In another embodiment of the present invention, one or more gas supply units 300′ can be provided and can be positioned in a plane direction parallel to the central axis of a chamber unit 100′ (refer to b) of FIG. 1).

The bioreactor 1a can further include a gas re-circulation line 330.

The gas re-circulation line 330 that is branched from the gas outflow channel 320 and is connected to the gas supply channel 310 is positioned outside the chamber unit 100. A part of gas flowing out from the gas outflow channel 320 to the outside of the chamber unit 100 can flow into the chamber unit 100 through the gas re-circulation line 330 and the gas supply channel 310.

In the embodiment of the present invention, the gas re-circulation line 330 can further include a peristaltic pump and valve (not illustrated) and can adjust a flow speed of gas which flows into the chamber unit 100 through the peristaltic pump and valve.

The gas can re-circulate through the gas re-circulation line 330, and the flow speed of gas surface can be increased through this re-circulation of the gas.

The bioreactor 1a can further include a sensor (not illustrated). The sensor can measure any one selected from the group consisting of pH, oxidation-reduction potential, dissolved oxygen, turbidity, a shape, and a combination thereof inside the chamber unit 100, and thus the sensor enables an optimal culture condition to be provided.

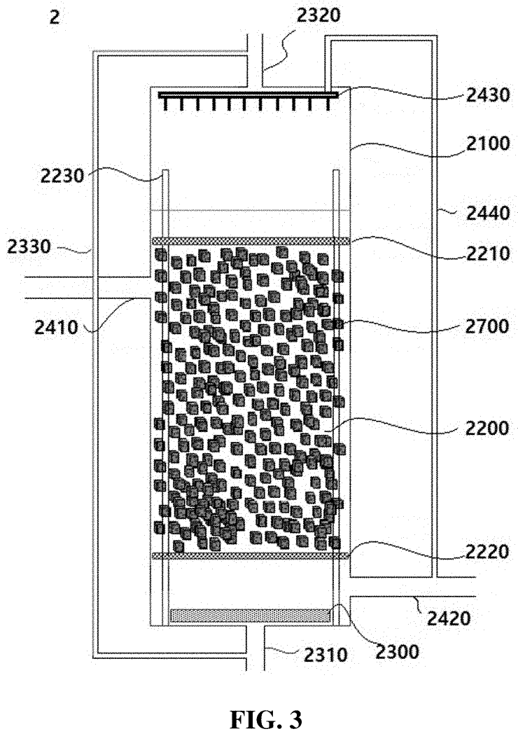

FIG. 3 is a schematic view of a bioreactor 2 according to still another embodiment of the present invention.

With reference to FIG. 3, the bioreactor 2 includes: a chamber unit 2100 having an accommodating space therein; an upper fixing member 2210 and a lower fixing member 2220 that are disposed at respective planes perpendicular to a central axis of the chamber unit 2100 inside the chamber unit 2100 and are movable in a central-axis direction of the chamber unit 2100; a filling layer 2200 that is positioned in a space between the upper fixing member 2210 and the lower fixing member 2220 inside the chamber unit 2100 and contains a plurality of carriers 2700; an inlet 2410 and an outlet 2420; and a liquid distributing unit 2430 that is positioned at an upper surface inside the chamber unit 2100.

Details of this embodiment corresponding to those of the above-described embodiments are replaced with the description provided in the above-described embodiments, and the following description focuses on details different from those of the above-described embodiments.

The liquid distributing unit 2430 is connected to a liquid vertical-circulation line 2440 outside the chamber unit 2100, is positioned at the upper side of the chamber unit 2100, and has a function of injecting a liquid such as a culture medium into the chamber unit 2100.

The liquid distributing unit 2430 can include a shower head unit such as a jet loop or an orifice tube which can uniformly inject a liquid to an internal cross-sectional area of the chamber unit 2100.

The bioreactor 2 can further include the liquid vertical-circulation line 2440 that is branched from the outlet 2420 and is connected to the liquid distributing unit 2430.

In the embodiment of the present invention, the culture medium injected through the inlet 2410 can be discharged outside the chamber unit 2100 through the outlet 2420.

At that time, a part of the culture medium discharged through the outlet 2420 can move through the liquid vertical-circulation line 2440 to the liquid distributing unit 2430 positioned at the upper side of the chamber unit 2100, can be re-injected by the liquid distributing unit 2430 into the chamber unit 2100, and can increase a gas-liquid substance transfer surface area by passing through the upper fixing member 2210 and the filling layer 2200 in this order such that productivity can be increased.

Specifically, the liquid can be injected into an upper side of the filling layer 2200 and can increase a liquid surface area while moving through surfaces of the carriers 2700, and thus the gas-liquid surface area can be increased.

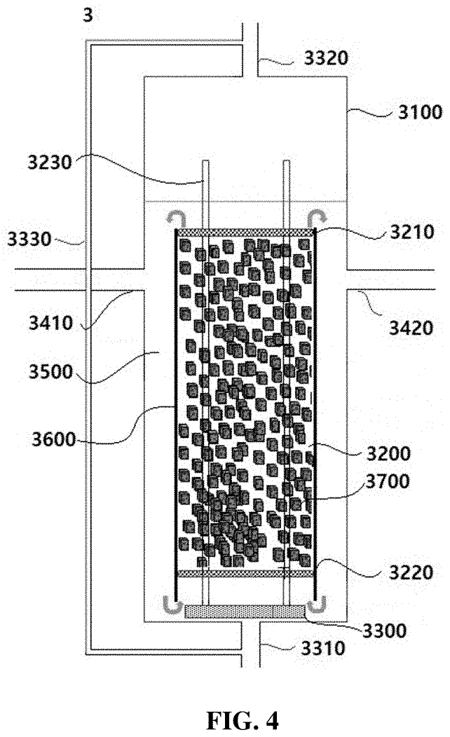

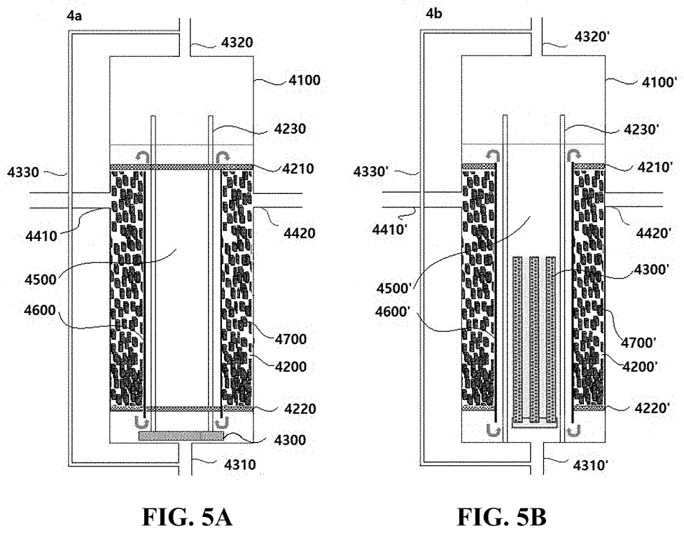

FIG. 4 is a schematic view of a bioreactor 3 according to still another embodiment of the present invention.

With reference to FIG. 4, the bioreactor 3 includes: a chamber unit 3100 having an accommodating space therein; a partition member 3600 that is positioned to be coaxial with a central axis of the chamber unit 3100 inside the chamber unit 3100 and is separated from a side wall of the chamber unit 3100 to form a circulation part 3500; an upper fixing member 3210 and a lower fixing member 3220 that are disposed at respective planes perpendicular to the central axis of the chamber unit 3100 and are positioned inside the partition member 3600; and a filling layer 3200 that is positioned in an inner space of the partition member 3600 and a space between the upper fixing member 3210 and the lower fixing member 3220 and contains a plurality of carriers 3700. The circulation part 3500 can be formed in a separated space between the side wall of the chamber unit 3100 and the partition member 3600.

Details of this embodiment corresponding to those of the above-described embodiments are replaced with the description provided in the above-described embodiments, and the following description focuses on details different from those of the above-described embodiments.

In the bioreactor 3 according to the embodiment of the present invention, the filling layer 3200 is positioned in the inner space of the partition member 3600 and the space between the upper fixing member 3210 and the lower fixing member 3220.

Since the partition member 3600 is positioned to be coaxial with the central axis of the chamber unit 3100 and is separated from the side wall of the chamber unit 3100 to form the circulation part 3500, it is desirable that a diameter of a cross section of the partition member 3600 be smaller than a diameter of a cross section of the chamber unit 3100.

In addition, since the filling layer 3200 is positioned inside the partition member 3600, the partition member can have a space which can accommodate the filling layer.

For reference, blue arrows in FIG. 4 represent flow of a liquid in the bioreactor 3. For example, flow of gas injected through a gas supply unit 3300 causes a culture medium to be raised inside the chamber unit 3100. The raised culture medium passes through the lower fixing member 3220, the filling layer 3200, and the upper fixing member 3210 in this order and then is dropped down through the circulation part 3500. The dropped culture medium moves to an inner side of the partition member 3600 and then is raised again due to the flow speed of gas injected through the gas supply unit 3300.

The circulation of the culture medium through the circulation part 3500 increases a retention time of fine bubbles in the filling layer 3200 and top and bottom mixtures of the culture medium, and thus homogeneity in the filling layer 3200 is increased such that a flow speed of the culture medium is increased, and the increased flow speed increases nutrient transfer and a nutrient diffusion rate.

FIG. 5 illustrates schematic views of bioreactors 4a and 4b according to still other embodiments of the present invention.

With reference to a) of FIG. 5, the bioreactor 4a includes: a chamber unit 4100 having an accommodating space therein; a partition member 4600 that is positioned to be coaxial with a central axis of the chamber unit 4100 inside the chamber unit 4100 and is separated from a side wall of the chamber unit 4100 to form a circulation part 4500; an upper fixing member 4210 and a lower fixing member 4220 that are disposed at respective planes perpendicular to the central axis of the chamber unit 4100 and are positioned in a space between an outer wall of the partition member 4600 and an inner wall of the chamber unit 4100; a filling layer 4200 that is formed in a space between the inner wall of the chamber unit 4100 and the outer wall of the partition member 4600 and a space between the upper fixing member 4210 and the lower fixing member 4220 and contains a plurality of carriers 4700. The circulation part 4500 can be formed in a space inside the partition member 4600.

Details of this embodiment corresponding to those of the above-described embodiments are replaced with the description provided in the above-described embodiments, and the following description focuses on details different from those of the above-described embodiments.

In the bioreactor 4a according to this embodiment of the present invention, the circulation part 4500 is positioned inside the partition member 4600.

For reference, blue arrows in FIG. 5 represent flow of a liquid in the bioreactors 4a and 4b. For example, flow of gas injected through a gas supply unit 4300 causes a culture medium to be raised inside the circulation part 4500. The raised culture medium moves to the upper fixing member 4210, then, passes through the upper fixing member 4210, and is dropped through the filling layer 4200. The culture medium out of the filling layer 4200 passes through the lower fixing member 4220 and then is raised through the circulation part 4500 due to flow of gas re-injected through the gas supply unit 4300.

According to still another aspect of the present invention, there is provided a method for culturing microorganisms by using the bioreactors according to the embodiments of the present invention.

The method for culturing microorganisms by using the bioreactor is performed by sterilizing an inside of the bioreactor containing carriers, filling the bioreactor with a sterilized new culture medium, then, performing inoculation with microorganisms to be cultured, and then operating the bioreactor.

The method for culturing microorganisms can be performed by any one method of batch culture, continuous culture, or semibatch culture.

In the method of batch culture, a culture medium is injected in an early stage, and then there is neither outflow nor inflow of a product or a culture medium while a reaction occurs. The method of batch culture is suitable for a test or a small-scale operation of a new process which is not completely developed, manufacturing of an expensive product, or processes which are difficult to change by continual operations.

The method of continuous culture is a method for culturing microorganisms by continuously injecting a new culture medium and discharging a culture medium in a reactor, the culture medium containing products and microorganisms. The continuous culture is advantageous in that growth of new microorganisms can be maximized.

The method of semibatch culture is a method for culturing microorganisms by injecting a certain volume of new culture medium periodically and discharging a culture medium in a reactor, the culture medium containing products. The method of semibatch culture can adjust a product concentration in a culture medium and can adjust a cell concentration inside a reactor, compared to the method of continuous culture, and thus the semibatch culture is advantageous in increasing a yield of products.

The bioreactors 1 to 4 of the present invention can perform the three methods for culturing microorganisms and can perform the microbiological culture by the method of semibatch culture.

### EMBODIMENTS

### Embodiment 1. Microbiological Culture

The microbiological culture was performed by the method of semibatch culture using the bioreactor according to the embodiment of the present invention, specifically, the bioreactor having the configuration illustrated in FIG. 1.

Clostridium autoethanogenum DSM10061 which is an anaerobic bacterium that produces ethanol in a carbon monoxide substrate condition was selected as a model strain, and a culture medium was prepared in accordance with a composition described in the following Table 1.

TABLE 1

| | | | | | |
|---|---|---|---|---|---|
| 20× Basal medium | 50 | ml | 1M MES | 10 | ml |
| NaCl | 18 | g/L | 1M K₂HPO₄ | 10 | ml |
| MgSO₄•7H₂O | 6.4 | g | Yeast extract | 2 | g |
| CaCl₂•2H₂O | 4 | g | Cysteine-HCl | 0.5 | g |
| NH₄Cl | 20 | g | 0.1% Resazurin | 0.1 | ml |
| | | | Deionized water | 920 | ml |
| 100× Microminerals | 10 | ml | 500× Vitamin | 2 | ml |
| | | | solution | | |
| Nitrilo acetic acid | 1.5 | g/L | | | |
| FeSO₄•7H₂O | 0.1 | g | | | |
| MnCl₂•4H₂O | 0.1 | g | | | |
| CoCl₂•6H₂O | 0.17 | g | | | |
| ZnCl₂ | 0.1 | g | | | |
| CaCl₂•6H₂O | 0.1 | g | | | |
| CuCl₂•2H₂O | 0.02 | g | | | |
| H₃BO₃ | 0.01 | g | | | |
| Na₂MoO₄ | 0.01 | g | | | |
| Na₂SeO₄ | 0.01 | g | | | |
| NaCl | 1 | g | | | |
| MnSO₄•H₂O | 1 | g | | | |
| Fe(NH₄)₂(SO₄)₂•6H₂O | 0.8 | g | | | |
| ZnSO₄•7H₂O | 0.2 | g | | | |
| NiCl₂•6H₂O | 0.02 | g | | | |
| Na₂WO₄ | 0.02 | g | | | |

A gas injecting device with holes having a diameter of 10 μm to 16 μm was installed on an inner bottom of the reactor. The reactor containing a culture medium and 24.12 g of a 1 cm³ polyurethane carrier and a container containing a medium were sterilized at 121° C. for 15 minutes. After the container containing the medium was cooled, 1% of K₂HPO₄ and 0.2% of a vitamin solution were injected in the medium.

An upper side of the reactor was subjected to flushing with syngas (8 to 2 as a ratio of CO to CO₂), and then 2 L of a new culture medium was supplied through a peristaltic pump.

After 60 ml of culture medium having a composition illustrated in Table 1 was put in a 160 ml airtight container, the rest volume was pressured with carbon monoxide by 1 atm., and Clostridium autoethanogenum DSM10061 was cultured at 37° C. and at 180 rpm. Then, 60 ml of nutrient solution containing mid-log cells was used as a reactor driving inoculum, a new culture medium was supplied at a ratio of 25% to 50% of the amount of culture medium inside the reactor every other day, without addition of an MES buffer solution during an operation of the bioreactor, and a fermented solution was obtained. During a period of culture, pH of the culture medium was adjusted with 4.6 N of NaOH, and a gas internal circulation speed was maintained at 50 rpm to 100 rpm using a peristaltic pump.

### Experimental Example 1. Cell Growth and Production Pattern of Product

Growth of a cell and products were observed by the method for culturing microorganisms according to Embodiment 1.

A cell concentration was obtained from an optical density at 600 nm using a UV-VIS spectrophotometer (V-730, Jasco, Japan).

Gas chromatography was performed using a thermal conductivity detector (GC-TCD, ACME6100, YL Instrument, Korea), thereby quantifying gas. In order to operate the thermal conductivity detector, an oven operating program and a setting condition were used.

Liquid metabolites were analyzed using gas chromatography by a flame ionization detector (GC-FID, ACME6100, YL Instrument, Korea). For the gas chromatography, 2 μl of sample solution was used, and standard solutions containing ethanol and acetic acid, respectively, were sequentially diluted and analyzed, and a black curve was obtained.

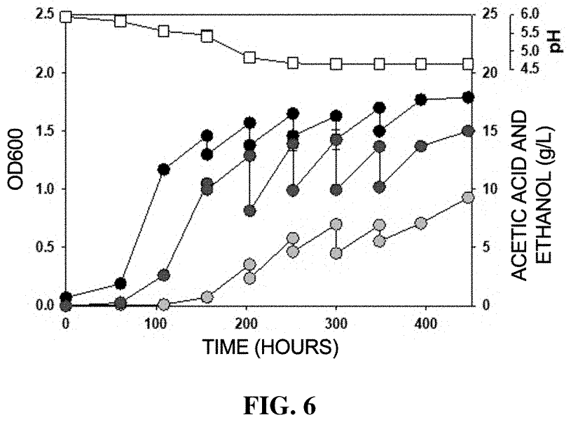

It was possible to observe that the growth of cells and production of products were steadily maintained even after the log phase from a result of the experiment (FIG. 6; black circle: growth pattern of cell; navy circle: production pattern of acetic acid; blue-green circle: production pattern of ethanol).

The description of the present invention described above is provided as an example, and a person of ordinary skill in the art to which the present invention pertains can understand that it is possible to easily modify the present invention to still another embodiment without changing the technical idea or an essential feature of the present invention. Therefore, the embodiments described above need to be understood as exemplified embodiments and not as embodiments to limit the present invention in every aspect. For example, configurational elements described in a single form can be realized in a distributed manner. Similarly, the configurational elements described in the distributed manner can be realized in a combined manner.

The scope of the present invention needs to be represented by the claims to be described below, and meaning and the scope of the claims and every modification or modified example derived from an equivalent concept of the claims need to be construed to be included in the scope of the present invention.

1. A bioreactor comprising:
a chamber unit having an accommodating space therein;
an upper fixing member and a lower fixing member that are disposed at respective planes perpendicular to a central axis of the chamber unit inside the chamber unit and are movable in a central-axis direction of the chamber unit; and
a filling layer that is formed in a space between the upper fixing member and the lower fixing member inside the chamber unit and contains a plurality of carriers.

2. The bioreactor according to claim 1,
wherein the filling layer has a volume which is adjustable by adjusting a position of the upper fixing member and/or the lower fixing member.

3. The bioreactor according to claim 1, further comprising:
a fixing-member adjusting unit.

4. The bioreactor according to claim 1, further comprising:
a gas supply unit.

5. The bioreactor according to claim 1, further comprising:
an inlet and an outlet.

6. The bioreactor according to claim 1, further comprising:
a gas supply channel and a gas outflow channel.

7. The bioreactor according to claim 6, further comprising:
a gas re-circulation line that is branched from the gas outflow channel and is connected to the gas supply channel.

8. The bioreactor according to claim 1, further comprising:
a sensor.

9. The bioreactor according to claim 8,
wherein the sensor measures any one selected from the group consisting of pH, oxidation-reduction potential, dissolved oxygen, turbidity, a shape, and a combination thereof.

10. The bioreactor according to claim 1, further comprising:
a liquid distributing unit that is positioned at an upper surface inside the chamber unit.

11. A bioreactor comprising:
a chamber unit having an accommodating space therein;
a partition member that is positioned to be coaxial with a central axis of the chamber unit inside the chamber unit and is separated from a side wall of the chamber unit to form a circulation part;
an upper fixing member and a lower fixing member that are disposed at respective planes perpendicular to the central axis of the chamber unit and are positioned inside the partition member; and
a filling layer that is formed in an inner space of the partition member and a space between the upper fixing member and the lower fixing member and contains a plurality of carriers.
wherein the circulation part is formed in a separated space between the side wall of the chamber unit and the partition member.

12. A bioreactor comprising:
a chamber unit having an accommodating space therein;
a partition member that is positioned to be coaxial with a central axis of the chamber unit inside the chamber unit and is separated from a side wall of the chamber unit to form a circulation part;
an upper fixing member and a lower fixing member that are disposed at respective planes perpendicular to the central axis of the chamber unit and are positioned in a space between an outer wall of the partition member and an inner wall of the chamber unit;
a filling layer that is formed in a space between the inner wall of the chamber unit and the outer wall of the partition member and a space between the upper fixing member and the lower fixing member and contains a plurality of carriers,
wherein the circulation part is formed in a space inside the partition member.

13. A method for culturing microorganisms by using the bioreactor according to claim 1.

14. The method for culturing microorganisms according to claim 13,
wherein the method for culturing microorganisms is performed by any one method of batch culture, continuous culture, or semibatch culture.
